(19) United States
(12) Patent Application Publication
(10) Pub. No.: US 2024/0417380 A1
(43) Pub. Date: Dec. 19, 2024

(54) CRYSTALLINE COMPOSITION, CRYSTAL OF BENZOXAZINE COMPOUND, METHOD FOR PRODUCING BENZOXAZINE COMPOUND, AND NOVEL COMPOUND

(71) Applicant: HONSHU CHEMICAL INDUSTRY CO., LTD., Chuo-ku, Tokyo (JP)

(72) Inventor: Daichi OKAMURA, Wakayama-shi (JP)

(21) Appl. No.: 18/701,542

(22) PCT Filed: Oct. 20, 2022
(86) PCT No.: PCT/JP2022/039120

§ 371 Date: Apr. 15, 2024

(30) Foreign Application Priority Data
Nov. 1, 2021 (JP) 2021-178424

(51) Int. Cl. C07D 265/16; C07C 217/90; C07C 251/24

(52) U.S. Cl. CPC C07D 265/16; C07C 217/90; C07C 251/24

(57) ABSTRACT

A crystalline composition containing a benzoxazine compound represented by general formula (1) is provided:

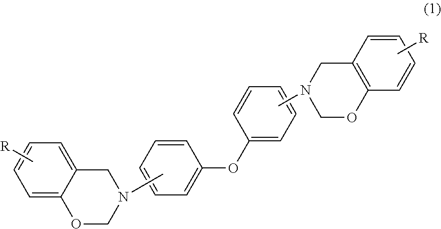

wherein each R is independently a hydrogen atom or a methyl group.

### TECHNICAL FIELD

The present invention relates to a crystalline composition containing a specific benzoxazine compound, a crystal of a benzoxazine compound, a method for producing a benzoxazine compound, and a novel compound that is an intermediate involved in the production method.

### BACKGROUND ART

Benzoxazine compounds are known as thermosetting resin raw materials that, when heated, undergo ring-opening polymerization of a benzoxazine ring to cure without producing any volatile by-products, and are used as raw materials of a material for an insulating substrate, a liquid crystal alignment agent, a resin for semiconductor sealing, and the like.

PTL 1 discloses a method in which a thermosetting composition containing compound (1-1-1) or/and compound (1-2-1) is handled in the form of a solution in acetone.

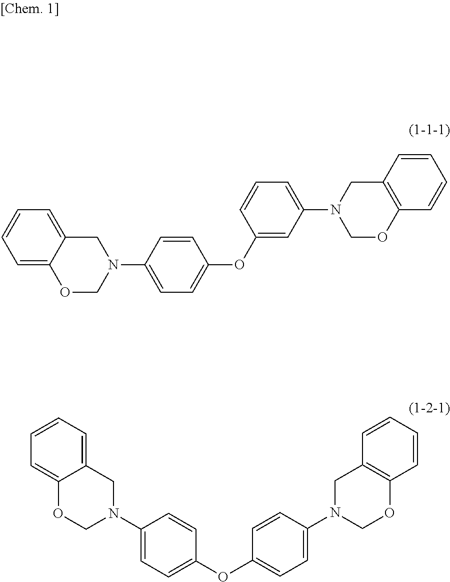 [SMILES: c1cc(Oc2ccc(N3COc4ccccc4C3)cc2)cc(N2COc3ccccc3C2)c1 ; c1ccc2c(c1)CN(c1ccc(Oc3ccc(N4COc5ccccc5C4)cc3)cc1)CO2]

PTL 2 discloses that in the synthesis of compound (1-1-1), the target obtained by subjecting a reaction solution to a post-treatment is isolated as a resinous substance and is about 60% pure.

PTL 3, which is a prior application filed by the present applicant, discloses that in the synthesis of compound (1-1-1), the target obtained by subjecting a reaction solution to a post-treatment was heated to melt, taken out of a reactor in the form of a molten liquid, solidified by cooling, and then pulverized to obtain a benzoxazine compound-containing composition.

### Citation List

### Patent Literature

PTL 1: Japanese Unexamined Patent Application Publication No. 2018-016684

PTL 2: Japanese Unexamined Patent Application Publication No. 2018-184533

PTL 3: Japanese Patent Application No. 2020-184536

### SUMMARY OF INVENTION

### Technical Problem

Benzoxazine compounds such as compound (1-1-1) and compound (1-2-1) have problems, for example, in that when they are taken out in the form of a solution, the volumetric efficiency in a reactor is reduced because a solvent is added, it is necessary to distill off a large amount of solvent when a cured product of a benzoxazine compound is obtained, and, furthermore, if the solvent remains, the solvent volatilizes at the time of heat curing to form voids in the cured product. There are also problems in that to take them out in melted form, an apparatus for heating and conservation of heat is required, a high temperature is required for melting, and in addition an operation such as pulverization of a resinous substance that has been solidified by cooling after being taken out is required for improving handleability.

In addition, since pulverized substances obtained by pulverizing the resinous substance adhere together again if stored at room temperature (about 20° C.), pulverization needs to be performed before each use, which is troublesome. To prevent this, storage at low temperature is necessary, but refrigeration equipment is required for storage and transport at low temperature.

As described above, there is a problem of inefficiency in the process of industrial production.

Furthermore, there is also a problem in that compound (1-1-1), when obtained by a production method known in the art, is about 70% pure and provides a composition having a low active ingredient content.

An object of the present invention is to provide means for solving the foregoing problems of the related art involved in benzoxazine compounds such as compound (1-1-1) and compound (1-2-1).

### Solution to Problem

To achieve the above object, the present inventors have conducted intensive studies and discovered a crystalline benzoxazine compound composition containing a benzoxazine compound represented by general formula (1), particularly, a crystal of a benzoxazine compound represented by general formula (1-1), the crystal having an endothermic peak top temperature in a specific range as determined by differential scanning calorimetry, thereby completing the present invention.

The present invention is as follows.
1. A crystalline composition containing a benzoxazine compound represented by general formula (1).

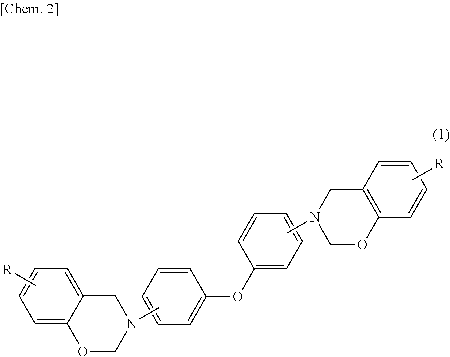

(In the formula, each R is independently a hydrogen atom or a methyl group.)
2. The crystalline composition according to 1., wherein a degree of crystallinity evaluated by X-ray diffractometry is 1% to 100%.
3. The crystalline composition according to 2., wherein the degree of crystallinity evaluated by X-ray diffractometry is 50% to 100%.
4. The crystalline composition according to 2., wherein the degree of crystallinity evaluated by X-ray diffractometry is 80% to 100%.
5. The crystalline composition according to 1., in which in a gel permeation chromatography measurement using a differential refractometer as a detector, a peak area of the benzoxazine compound represented by general formula (1) is in a range of 80 area % to 100 area % relative to a peak area of all components detected.
6. The crystalline composition according to 1., wherein a maximum endothermic peak temperature determined by differential scanning calorimetry is 90° C. to 120° C.
7. A crystal of a benzoxazine compound represented by general formula (1-1), wherein a maximum endothermic peak temperature determined by differential scanning calorimetry is 90° C. to 120° C.

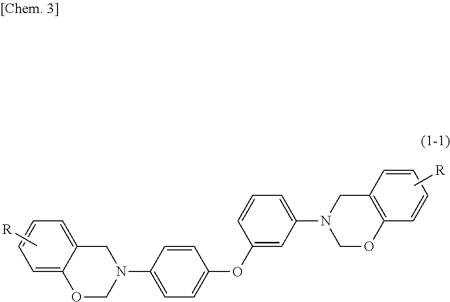

(In the formula, each R is independently a hydrogen atom or a methyl group.)
8. The crystal according to 7., wherein the benzoxazine compound represented by general formula (1-1) is a benzoxazine compound represented by formula (1-1-1).

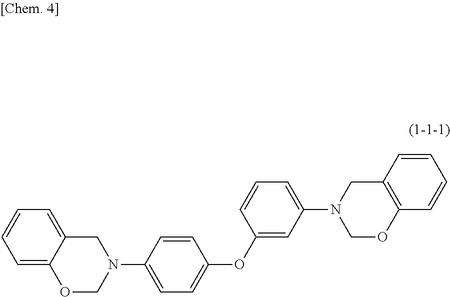

9. The crystal of the benzoxazine compound according to 7.,wherein in a gel permeation chromatography measurement using a differential refractometer as a detector, a peak area of the benzoxazine compound represented by general formula (1-1) is in a range of 80 area % to 100 area % relative to a peak area of all components detected.
10. A method for producing a benzoxazine compound represented by general formula (1), the method including three reaction steps of a first step to a third step.

### First Step

A step of reacting a diaminodiphenyl ether represented by general formula (2) and a hydroxybenzaldehyde represented by general formula (3) to obtain a compound represented by general formula (A).

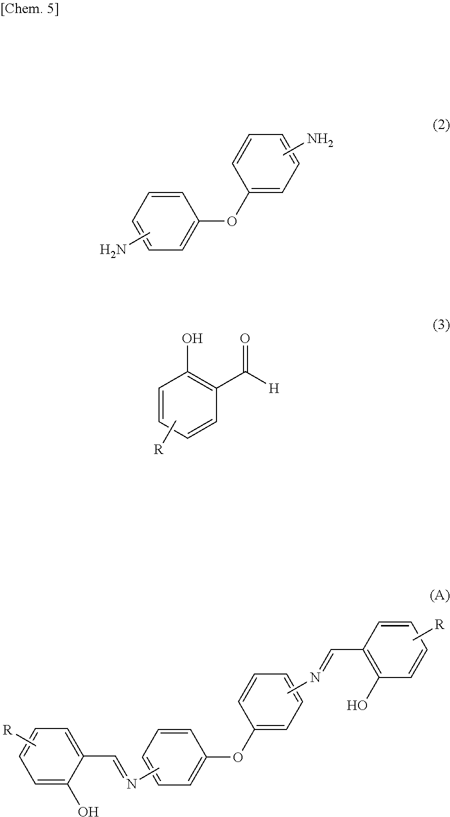

(In the formula, each R is independently a hydrogen atom or a methyl group.)

### Second Step

A step of reducing the compound represented by general formula (A) to obtain a compound represented by general formula (B).

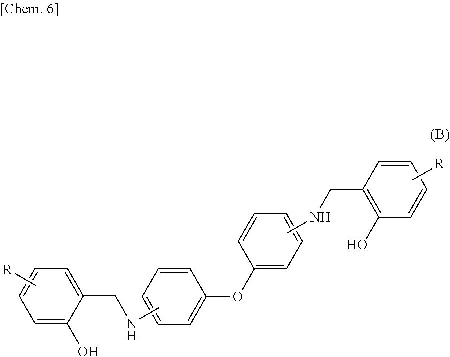

(In the formula, each R is independently a hydrogen atom or a methyl group.)

### Third Step

A step of reacting the compound represented by general formula (B) and a formaldehyde to obtain a compound represented by general formula (1).

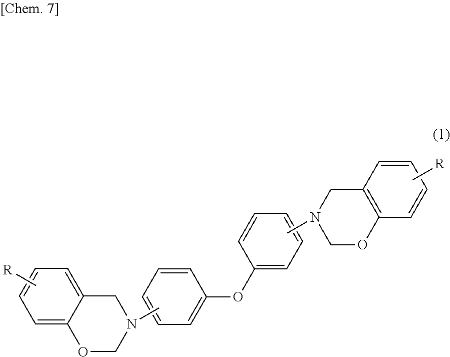

(In the formula, each R is independently a hydrogen atom or a methyl group.)
11. A compound represented by general formula (A-1).

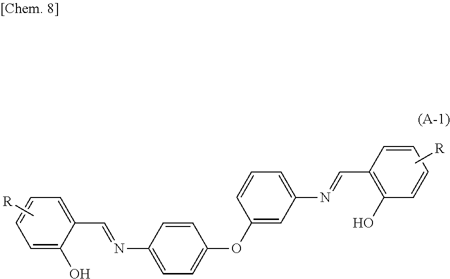

(In the formula, each R is independently a hydrogen atom or a methyl group.)
12. A compound represented by general formula (B-1).

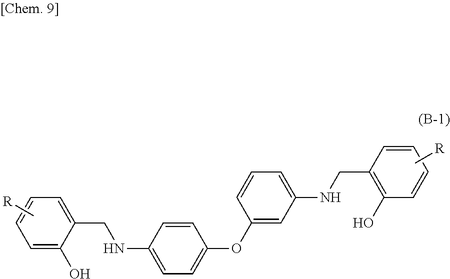

(In the formula, each R is independently a hydrogen atom or a methyl group.)

### Advantageous Effects of Invention

The crystalline composition containing a benzoxazine compound represented by general formula (1) and the crystal of a benzoxazine compound represented by general formula (1-1) according to the present invention are crystalline, and thus are very easy to handle and have high storage stability.

Furthermore, in a more preferred embodiment, the purity is higher than those of compositions containing benzoxazine compounds known in the art, and active ingredients are contained in larger amounts, which is useful in producing cured products using them.

That is, provision of the crystalline composition and the crystal of the benzoxazine compound according to the present invention solves the problems of the related art and contributes to improving the efficiency in the process of industrial production and in use, and thus is very useful. The production method according to the present invention can produce the crystalline composition and the crystal of the benzoxazine compound described above, and thus is very useful. In addition, the method can produce the benzoxazine compound represented by general formula (1) with high purity, and is very useful.

The compound represented by general formula (A-1) according to the present invention and the compound represented by general formula (B-1) according to the present invention are intermediates of some of the compounds obtained by the production method according to the present invention, and are very useful for producing the crystalline composition and the crystal of the benzoxazine compound described above.

### BRIEF DESCRIPTION OF DRAWINGS

FIG. 1 shows a chart of gel permeation chromatography analysis of a composition containing compound (B-1-1) obtained in Example 1.

FIG. 2 shows a chart of X-ray diffraction (XRD) measurement of a compound (1-1-1)-containing composition C1 obtained in Example 1.

FIG. 3 shows a chart of gel permeation chromatography analysis of the compound (1-1-1)-containing composition C1 obtained in Example 1.

FIG. 4 shows a chart of differential scanning calorimetry (DSC) data of the compound (1-1-1)-containing composition C1 obtained in Example 1.

FIG. 5 shows a chart of X-ray diffraction (XRD) measurement of a compound (1-1-1)-containing composition C2 obtained in Example 2.

FIG. 6 shows a chart of gel permeation chromatography analysis of the compound (1-1-1)-containing composition C2 obtained in Example 2.

FIG. 7 shows a chart of differential scanning calorimetry (DSC) data of the compound (1-1-1)-containing composition C2 obtained in Example 2.

FIG. 8 shows a chart of X-ray diffraction (XRD) measurement of a compound (1-1-1)-containing composition C3 obtained in Comparative Example 1.

FIG. 9 shows a chart of gel permeation chromatography analysis of the compound (1-1-1)-containing composition C3 obtained in Comparative Example 1.

FIG. 10 shows a chart of differential scanning calorimetry (DSC) data of the compound (1-1-1)-containing composition C3 obtained in Comparative Example 1.

FIG. 11 shows a chart of X-ray diffraction (XRD) measurement of a compound (1-1-1)-containing composition C4 obtained in Comparative Example 2.

FIG. 12 shows a chart of gel permeation chromatography analysis of the compound (1-1-1)-containing composition C4 obtained in Comparative Example 2.

FIG. 13 shows a chart of X-ray diffraction (XRD) measurement of a compound (1-1-1)-containing composition C5 obtained in Comparative Example 3.

FIG. 14 shows a chart of gel permeation chromatography analysis of the compound (1-1-1)-containing composition C5 obtained in Comparative Example 3.

FIG. 15 shows photographs of the compound (1-1-1)-containing composition C1 obtained in Example 1 in “Test for assessing storage stability at high temperatures and handleability of crystal”, the photographs showing a state where the composition C1 is placed in a sample bottle and capped (15-1) and states where the composition C1 is placed in sample bottles and the sample bottles are inverted after being stored for 24 hours at predetermined temperatures of 40° C. (15-2), 50° C. (15-3), and 60° C. (15-4).

FIG. 16 shows photographs of the compound (1-1-1)-containing composition C3 obtained in Comparative Example 1 in “Test for assessing storage stability at high temperatures and handleability of crystal”, the photographs showing a state where the composition C3 is placed in a sample bottle and capped (16-1) and states where the composition C3 is placed in sample bottles and the sample bottles are inverted after being stored for 24 hours at predetermined temperatures of 40° C. (16-2), 50° C. (16-3), and 60° C. (16-4).

FIG. 17 shows photographs of the compound (1-1-1)-containing composition C4 obtained in Comparative Example 2 in “Test for assessing storage stability at high temperatures and handleability of crystal”, the photographs showing a state where the composition C4 is placed in a sample bottle and capped (17-1) and states where the composition C4 is placed in sample bottles and the sample bottles are inverted after being stored for 24 hours at predetermined temperatures of 40° C. (17-2), 50° C. (17-3), and 60° C. (17-4).

FIG. 18 shows photographs of the compound (1-1-1)-containing composition C5 obtained in Comparative Example 3 in “Test for assessing storage stability at high temperatures and handleability of crystal”, the photographs showing a state where the composition C5 is placed in a sample bottle and capped (18-1) and states where the composition C5 is placed in sample bottles and the sample bottles are inverted after being stored for 24 hours at predetermined temperatures of 40° C. (18-2), 50° C. (18-3), and 60° C. (18-4).

### DESCRIPTION OF EMBODIMENTS

A crystalline composition according to the present invention contains a benzoxazine compound represented by general formula (1).

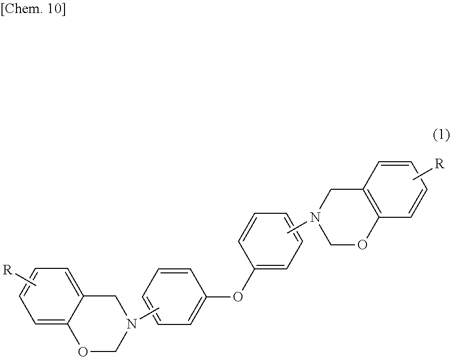

(In the formula, each R is independently a hydrogen atom or a methyl group.)

The degree of crystallinity of the crystalline composition according to the present invention as evaluated by X-ray diffractometry is preferably 1% to 100%, more preferably 50% to 100%, still more preferably 80% to 100%, particularly preferably 90% to 100%.

The degree of crystallinity was evaluated, on the basis of a powder X-ray diffraction pattern obtained through measurement by X-ray diffractometry, by performing the calculation of the following known formula. This calculation can be performed using, for example, SmartLab Studio II software manufactured by Rigaku Corporation.

Degree of crystallinity=crystalline peak area÷(crystalline peak area+amorphous peak area)×100

For the content of the benzoxazine compound represented by general formula (1) in the crystalline composition according to the present invention, in a gel permeation chromatography measurement using a differential refractometer as a detector, the peak area of the benzoxazine compound represented by general formula (1) is preferably in the range of 80 area % to 100 area %, more preferably 85 area % to 100 area %, still more preferably 87 area % to 100 area %, particularly preferably 90 area % to 100 area %, relative to the peak area of all components detected. R in the benzoxazine compound represented by general formula (1) is a hydrogen atom or a methyl group, preferably a hydrogen atom.

The benzoxazine compound represented by general formula (1) contained in the crystalline composition according to the present invention may be a single compound or may include a plurality of compounds, but is preferably a single compound.

The benzoxazine compound represented by general formula (1) includes benzoxazine compounds represented by general formulae (1-1), (1-2), and (1-3).

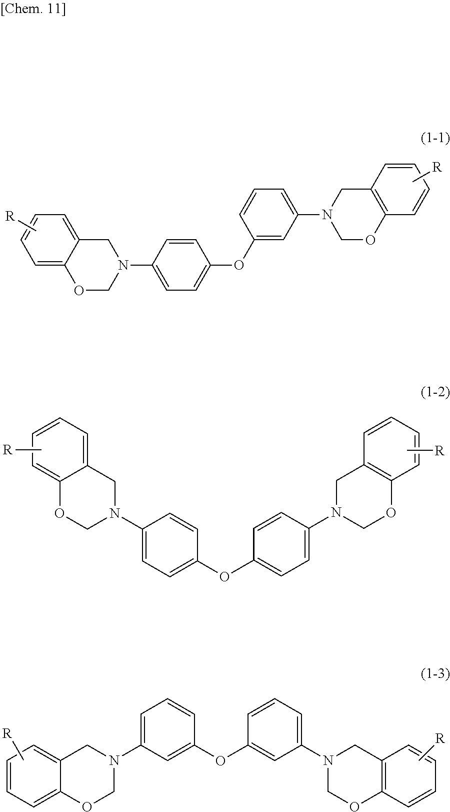

(In the formulae, each R is independently a hydrogen atom or a methyl group.)

Of these, the benzoxazine compound represented by general formula (1-1) is particularly preferred from the viewpoint of obtaining a resin having high heat resistance.

Specific examples of the benzoxazine compound represented by general formula (1) include compounds represented by the following chemical structural formulae.

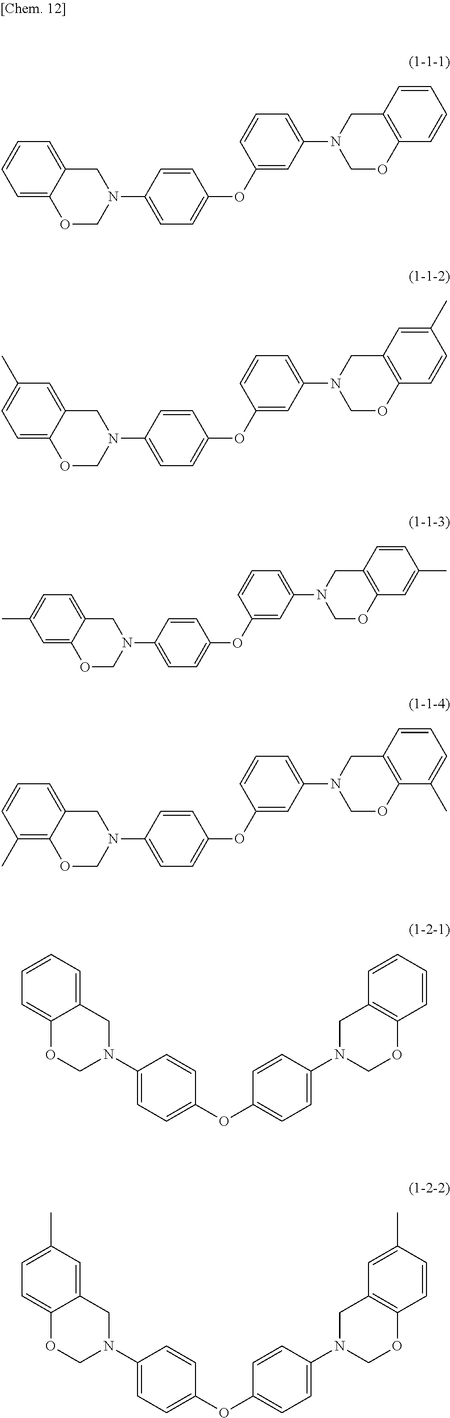

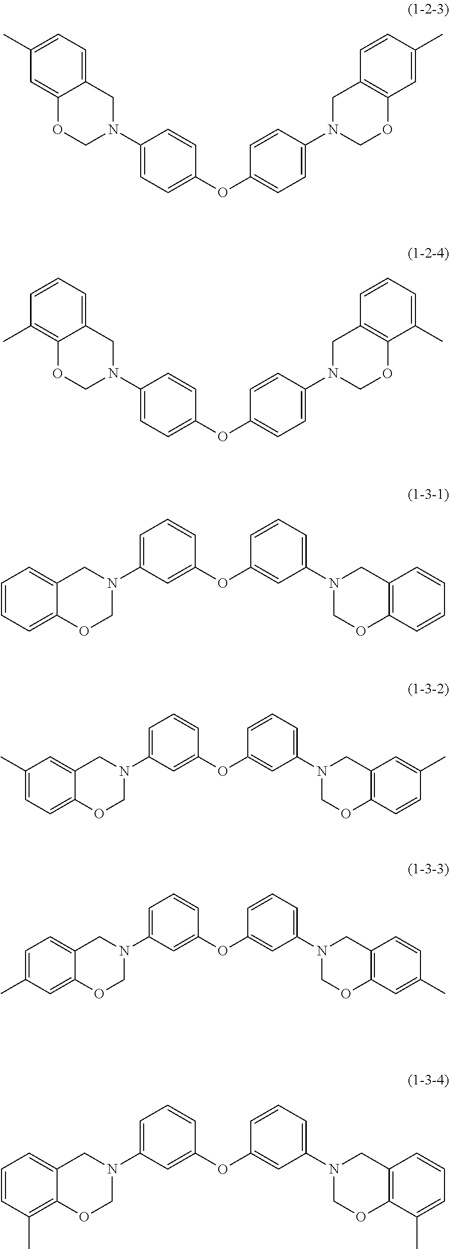

Compounds (1-1-1) to (1-1-4) are specific examples of the benzoxazine compound represented by general formula (1-1), compounds (1-2-1) to (1-2-4) are specific examples of the benzoxazine compound represented by general formula (1-2), and compounds (1-3-1) to (1-3-4) are specific examples of the benzoxazine compound represented by general formula (1-3).

Of these, from the viewpoint of obtaining a resin having high heat resistance, compounds (1-1-1) to (1-1-4) are preferred, and compound (1-1-1) is particularly preferred.

The maximum endothermic peak temperature of the crystalline composition according to the present invention as determined by differential scanning calorimetry is preferably 90° C. to 120° C., more preferably in the range of 95° C. to 115° C., still more preferably in the range of 100° C. to 110° C. This endothermic peak indicates that a melting phase change of the benzoxazine compound from a crystal phase to a liquid phase has occurred. It is presumed that the crystalline composition according to the present invention produces the advantageous effects of the present invention because of having a crystal structure that undergoes this phase change.

A crystal of a benzoxazine compound according to the present invention relates to a benzoxazine compound represented by general formula (1-1). Specific examples thereof include compounds (1-1-1) to (1-1-4), and compound (1-1-1) is particularly preferred.

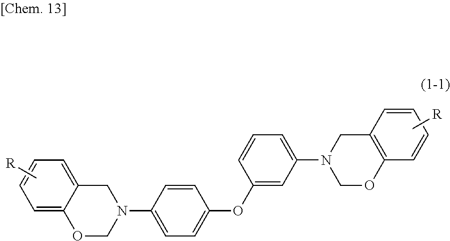

(In the formula, each R is independently a hydrogen atom or a methyl group.)

The maximum endothermic peak temperature of the crystal of the benzoxazine compound according to the present invention as determined by differential scanning calorimetry is in the range of 90° C. to 120° C., preferably in the range of 95° C. to 115° C., more preferably in the range of 100° C. to 110° C. This endothermic peak indicates that a melting phase change of the benzoxazine compound from a crystal phase to a liquid phase has occurred. It is presumed that the crystal according to the present invention produces the advantageous effects of the present invention because of having a crystal structure that undergoes this phase change.

The purity of the crystal of the benzoxazine compound according to the present invention based on gel permeation chromatography analysis is preferably 80% or more, more preferably 85% or more, still more preferably 87% or more, particularly preferably 90% or more.

The purity based on gel permeation chromatography analysis means the percentage of the peak area of the benzoxazine compound represented by general formula (1-1) relative to the peak area of all components detected when the crystal of the benzoxazine compound is measured by gel permeation chromatography using a differential refractometer as a detector.

The crystal of the benzoxazine compound according to the present invention preferably exhibits crystallinity as determined from a diffraction pattern by X-ray diffractometry. The degree of crystallinity evaluated by X-ray diffractometry is preferably 1% to 100%, more preferably 50% to 100%, still more preferably 80% to 100%, particularly preferably 90% to 100%.

The crystalline composition according to the present invention and the crystal of the benzoxazine compound according to the present invention are produced by a production method including the following three reaction steps of a first step to a third step.

### First Step

A step of reacting a diaminodiphenyl ether represented by general formula (2) and a hydroxybenzaldehyde represented by general formula (3) to obtain a compound represented by general formula (A).

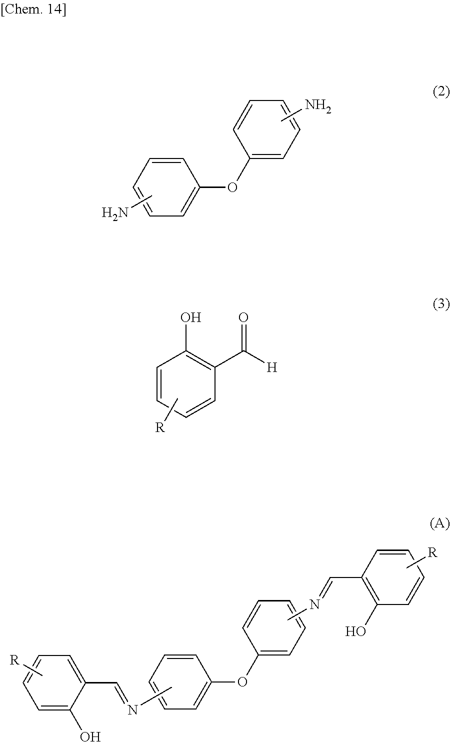

(In the formula, each R is independently a hydrogen atom or a methyl group.)

Here, from the viewpoint of obtaining a benzoxazine compound represented by general formula (1-1) that can provide a resin having high heat resistance, it is preferable to use 3,4′-diaminodiphenyl ether as the diaminodiphenyl ether represented by general formula (2), in which case among the compounds represented by general formula (A), a compound represented by general formula (A-1) can be obtained.

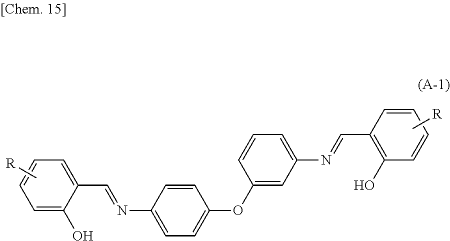

(In the formula, each R is independently a hydrogen atom or a methyl group.)

### Second Step

A step of reducing the compound represented by general formula (A) to obtain a compound represented by general formula (B).

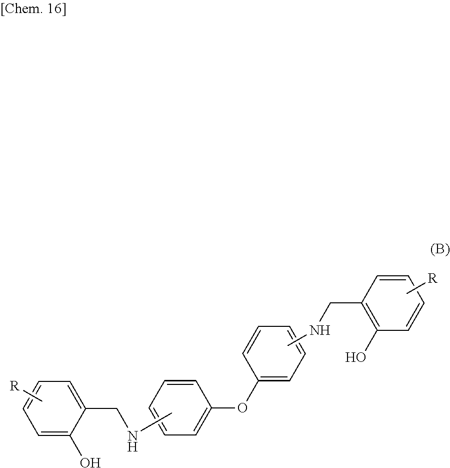

(In the formula, each R is independently a hydrogen atom or a methyl group.)

Here, from the viewpoint of obtaining a benzoxazine compound represented by general formula (1-1) that can provide a resin having high heat resistance, it is preferable to use the compound represented by general formula (A-1) as the compound represented by general formula (A), in which case a compound represented by general formula (B-1) can be obtained.

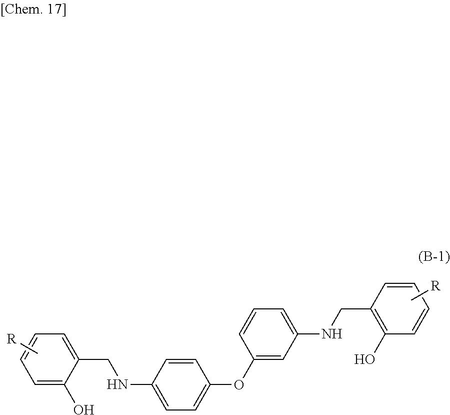

(In the formula, each R is independently a hydrogen atom or a methyl group.)

### Third Step

A step of reacting the compound represented by general formula (B) and a formaldehyde to obtain a compound represented by general formula (1).

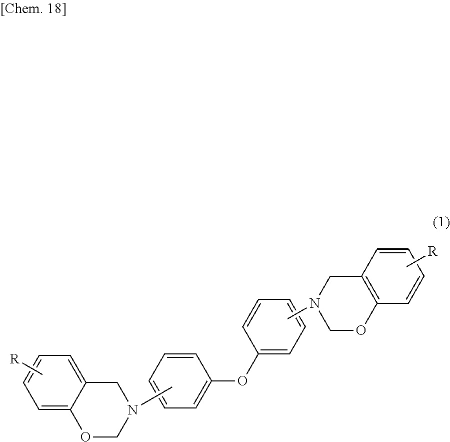

(In the formula, each R is independently a hydrogen atom or a methyl group.)

The method for producing a benzoxazine compound in the present invention will be described in detail below taking compound (1-1-1) as an example. As shown by the reaction formulae, the benzoxazine compound in the present invention is produced through three reaction steps of first to third steps.

### First Step

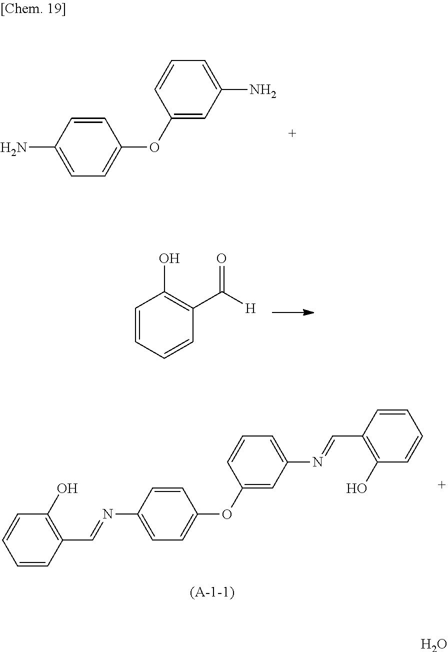 [SMILES: Nc1ccc(Oc2cccc(N)c2)cc1 ; O=Cc1ccccc1O ; Oc1ccccc1/C=N/c1ccc(Oc2cccc(/N=C/c3ccccc3O)c2)cc1 ; O]

### Second Step

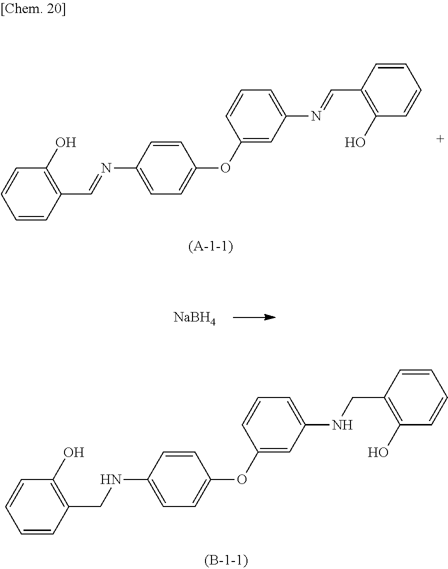 [SMILES: Oc1ccccc1/C=N/c1ccc(Oc2cccc(/N=C/c3ccccc3O)c2)cc1 ; "NaBH4" ; Oc1ccccc1CNc1ccc(Oc2cccc(NCc3ccccc3O)c2)cc1]

### Third Step

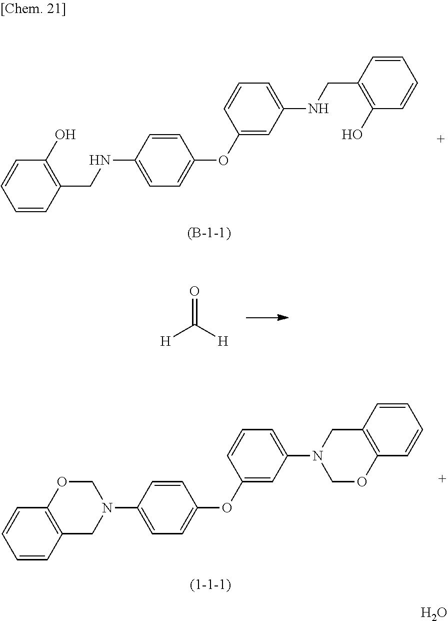 [SMILES: Oc1ccccc1CNc1ccc(Oc2cccc(NCc3ccccc3O)c2)cc1 ; C=O ; c1cc(Oc2ccc(N3COc4ccccc4C3)cc2)cc(N2COc3ccccc3C2)c1 ; O]

### First Step

As shown by the reaction formula, the first step is a step of obtaining compound (A-1-1) through condensation reaction of 3,4′-diaminodiphenyl ether and 2-hydroxybenzaldehyde.

The molar ratio between 3, 4′-diaminodiphenyl ether and 2-hydroxybenzaldehyde used, as expressed as 3,4′-diaminodiphenyl ether/2-hydroxybenzaldehyde, is preferably in the range of 1.0/1.6 to 1.0/4.0, more preferably in the range of 1.0/2.0 to 1.0/2.5.

A method in which 2-hydroxybenzaldehyde is added in the presence of 3,4′-diaminodiphenyl ether and a solvent is preferred, and 2-hydroxybenzaldehyde may be added in one portion or may be added dropwise over several minutes to several hours. Water produced may be but need not be distilled off. A catalyst for accelerating the reaction is not particularly necessary.

The solvent for use is preferably a lower aliphatic alcohol, an aromatic hydrocarbon, an ether, or a saturated aliphatic hydrocarbon, and in particular, water, methanol, ethanol, 1-propanol, 2-propanol, toluene, xylene, tetrahydrofuran, dioxolane, hexane, heptane, cyclohexane, or the like is suitable. The amount of solvent used is typically in the range of 50 to 2000 parts by weight, preferably in the range of 100 to 1500 parts by weight, relative to 100 parts by weight of 3, 4′-diaminodiphenyl ether.

The reaction temperature is preferably in the range of 20° C. to 90° C., more preferably in the range of 40° C. to 80° C. The reaction pressure may be any of normal pressure, reduced pressure, or increased pressure.

After completion of the reaction, the resulting solution may proceed to the subsequent second step without any additional treatment, but filtration and drying may be performed to obtain a solid, or after the solid matter in the reaction solution is dissolved, washing with water, concentration, crystallization, filtration, and drying may be performed to obtain solid matter.

### Second Step

As shown by the reaction formula, the second step is a step of reducing compound (A-1-1) obtained in the first step with a reducing agent such as sodium borohydride, lithium aluminum hydride, sodium cyanoborohydride, or lithium borohydride to obtain compound (B-1-1).

For the reducing agent used, the molar ratio of compound (A-1-1)/reducing agent is preferably in the range of 1.0/0.5 to 1.0/4.0, more preferably in the range of 1.0/1.0 to 1.0/3.0.

The reducing agent is preferably added in the presence of compound (A-1-1) and a solvent, and the reducing agent may be added in one portion or may be added over several minutes to several hours.

A catalyst for accelerating the reaction is not particularly necessary, but an acid catalyst or a base catalyst can be used as needed.

The solvent for use is preferably a lower aliphatic alcohol, and in particular, methanol, ethanol, 1-propanol, or 2-propanol is suitable. The amount of solvent used is typically in the range of 50 to 2000 parts by weight, preferably in the range of 100 to 1500 parts by weight, relative to 100 parts by weight of compound (A-1-1).

The reaction temperature is preferably in the range of −20° C. to 80° C., more preferably in the range of 0° C. to 40° C. The reaction pressure may be any of normal pressure, reduced pressure, or increased pressure.

After completion of the reaction, it is preferable to subject the resulting slurry liquid to solid-liquid separation by filtration to obtain a solid, then wash the obtained solid with water, a lower aliphatic alcohol, a water-mixed solvent thereof, or the like, and perform drying under reduced pressure.

### Third Step

As shown by the reaction formula, the third step is a step of performing a cyclization reaction using compound (B-1-1) obtained in the second step and formaldehyde or a formaldehyde such as an aqueous formaldehyde solution, 1,3,5-trioxane, or paraformaldehyde to obtain compound (1-1-1).

For formaldehyde used, the molar ratio of compound (B-1-1)/formaldehyde is preferably in the range of 1.0/1.7 to 1.0/4.0, more preferably in the range of 1.0/2.0 to 1.0/3.5.

It is preferable to add formaldehyde in the presence of compound (B-1-1) and a solvent, and formaldehyde may be added in one portion or may be added over several minutes to several hours. Water produced may be but need not be distilled off.

A catalyst for accelerating the reaction is not particularly necessary, but an acid catalyst or a base catalyst can be used as needed. In this case, examples of acid catalysts that can be used include, but are not limited to, concentrated hydrochloric acid, hydrochloric acid gas, trifluoroacetic acid, methanesulfonic acid, p-toluenesulfonic acid, benzoic acid, and mixtures thereof, and examples of base catalysts that can be used include, but are not limited to, sodium hydroxide, sodium carbonate, triethylamine, triethanolamine, and mixtures thereof.

The solvent for use is preferably a lower aliphatic alcohol, an aromatic hydrocarbon, an ether, or a saturated aliphatic hydrocarbon, and in particular, water, methanol, ethanol, 1-propanol, 2-propanol, toluene, xylene, tetrahydrofuran, dioxolane, hexane, heptane, cyclohexane, or the like is suitable. The amount of solvent used is typically in the range of 50 to 2000 parts by weight, preferably in the range of 100 to 1500 parts by weight, relative to 100 parts by weight of compound (B-1-1).

The reaction temperature is preferably in the range of 20° C. to 100° C., more preferably in the range of 20° C. to 70° C. The reaction pressure may be any of normal pressure, reduced pressure, or increased pressure.

After completion of the reaction, the solvent is concentrated from the resulting solution, and the amount of the solvent after the concentration is typically in the range of 30 to 1000 parts by weight, preferably in the range of 50 to 500 parts by weight, relative to 100 parts by weight of compound (B-1-1).

Thereafter, cooling is performed, and the resulting solid is subjected to solid-liquid separation by filtration to obtain a solid. The solid obtained is dried under reduced pressure.

### EXAMPLES

The present invention will now be described more specifically with reference to Examples.

Values of physical properties in the following examples were determined by the following methods.

### Analysis Methods

### 1. Differential Scanning Calorimetry (DSC)

A crystalline composition/a crystal of a benzoxazine compound in an amount of 2.5 to 3.5 mg was accurately weighed into an aluminum pan, hermetically sealed, and measured using an apparatus with aluminum oxide as a control under measurement conditions.

Apparatus: DSC7020/manufactured by Hitachi High-Tech Science Corporation

### Measurement Conditions

Heating rate: 10° C./min

Measurement temperature range: 30° C. to 300° C.

Measurement atmosphere: nitrogen, 50 mL/min

### 2. Gel Permeation Chromatography (Purity Analysis)

A 600-fold diluted solution of 10 mg of a crystalline composition/a crystal of a benzoxazine compound in tetrahydrofuran was measured using an apparatus under the following measurement conditions.

Apparatus: HLC-8320/manufactured by Tosoh Corporation

Detector: differential refractometer (RI)

### Measurement Conditions

Flow rate: 1 mL/min

Eluate: tetrahydrofuran

Temperature: 40° C.

Wavelength: 254 nm

Sampling pitch: 100 msec

Injection volume: 20 μL

### Column (From Upstream)

Guard Column HXL-L+G4000HXL+G3000HXL+G2000HXL×2 (7.8 mm ID×30 cm, manufactured by Tosoh Corporation)

### 3. Confirmation of Crystallinity and Evaluation of Degree of Crystallinity (X-Ray Diffractometry: XRD)

Each of the benzoxazine compounds obtained in Examples and Comparative Examples in an amount of 0.1 g was loaded in a sample loading section of a glass test plate and measured using the following apparatus under the following conditions.

### Measurement Apparatus

MiniFlex600-C/manufactured by Rigaku Corporation

### Measurement Conditions

X-ray source: CuKα

Scan axis: 2θ/θ

Mode: continuous

Measurement range: 2θ=5° to 90°

Step: 0.02°

Speed measurement time: 10°/min

Entrance slit: 0.25°

Receiving slit: 13.00 mm

Tube voltage: 40 kV

Tube current: 15 mA

### Evaluation of Degree of Crystallinity

The degree of crystallinity was evaluated, on the basis of a powder X-ray diffraction pattern obtained through measurement by the above-described method, by performing the calculation of the following known formula using SmartLab Studio II software manufactured by Rigaku Corporation.

Degree of crystallinity=crystalline peak area÷(crystalline peak area+amorphous peak area)×100

### Example 1

In a four-necked flask equipped with a thermometer, a stirrer, and a condenser, 127 g (0.64 mol) of 3,4′-diaminodiphenyl ether and 635 g of ethanol were loaded, and after the reaction vessel was purged with nitrogen, 168 g (1.38 mol) of 2-hydroxybenzaldehyde was added dropwise at 40° C. over 30 minutes. Thereafter, 630 g of ethanol was added, and stirring was performed at 60° C. for 2 hours and at 78° C. under reflux for 8 hours (first step). ¹H-NMR analysis confirmed that compound (A-1-1) was produced and contained in the resulting reaction solution. The ¹H-NMR data of compound (A-1-1) is shown.

¹H-NMR (400 MHZ) measurement (solvent: CDCl₃): 6.87 to 7.11 (m, 10H), 7.22 to 7.40 (m, 6H), 8.57 (s, 1H), 8.60 (s, 1H).

After completion of the reaction, the reaction solution was cooled to 25° C., 1138 g of ethanol was added, and 53 g (1.39 mol) of sodium borohydride was intermittently added over 2 hours. Thereafter, stirring was performed at 25° C. for 7 hours. After completion of the reaction, 1510 g of water was added, and stirring was performed at 25° C. for 14hours. The resulting slurry liquid was subjected to solid-liquid separation by filtration to obtain a solid. The solid obtained was washed with 300 g of a 30% aqueous methanol solution twice and with 500 g of water, and then dried at 50° C. under reduced pressure to obtain 280 g of a solid of compound (B-1-1) (second step). ¹H-NMR analysis confirmed that the solid obtained was compound (B-1-1). The ¹H-NMR data of compound (B-1-1) is shown.

¹H-NMR (400 MHZ) measurement (solvent: CDCl₃): 4.18 (d, 4H), 6.20 (s, 1H), 6.26 (d, 1H), 6.33 (d, 1H), 6.53 to 6.87(m, 8H), 6.92 to 7.13 (m, 5H).

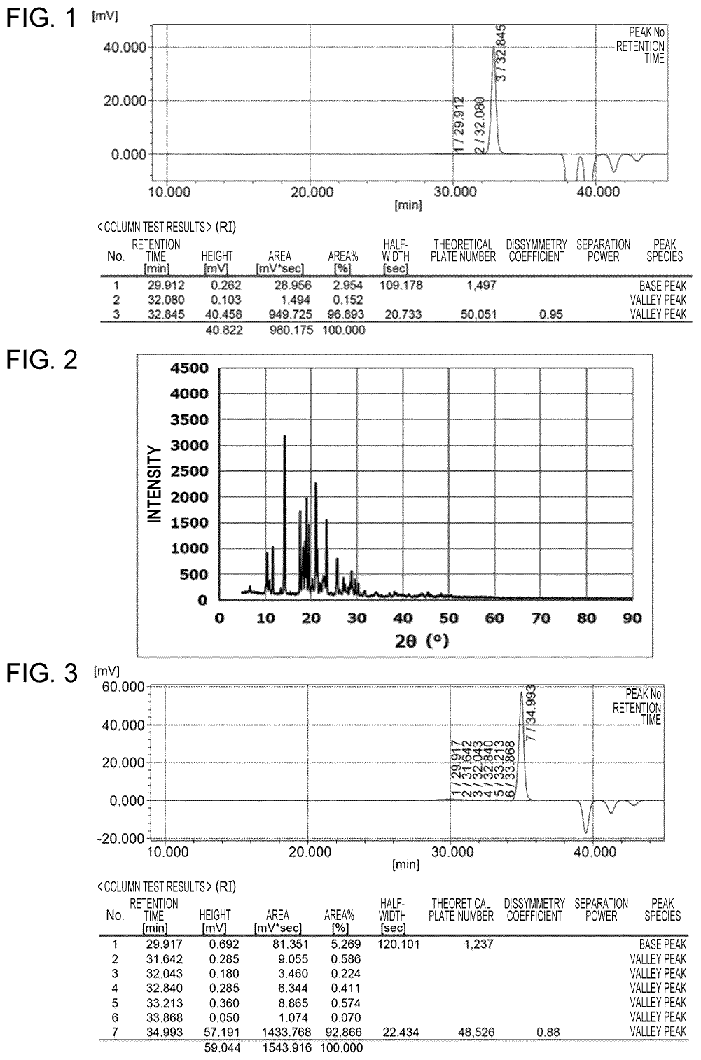

The obtained composition containing compound (B-1-1) had a purity of 96.9 area % as determined by gel permeation chromatography analysis using a differential refractometer as a detector. A chart of this gel permeation chromatography analysis is shown in FIG. 1.

Subsequently, 280 g of the composition containing compound (B-1-1) obtained in the second step, 2290 g of butyl acetate, 47 g (0.7 mol, 1.1 times the amount of 3,4-diaminodiphenyl ether on a molar basis) of acetic acid, and 490 g of water were loaded in a four-necked flask, stirred at 70° C. for 2 hours, and then allowed to stand, and an aqueous layer was separated and removed. The oil layer obtained was adjusted to 70° C. After 500 g of water was added with stirring, the resulting mixture was stirred for 30 minutes and allowed to stand, and an aqueous layer was separated and removed. The procedure from the addition of water to the extraction of an aqueous layer was repeated four times. The final pH of the oil layer was 3.

The oil layer obtained was cooled to 40° C., and at 40° C., 207 g (2.4 mol) of 35% formalin was added dropwise over 30 minutes. Stirring was performed at 40° C. for 5 hours (third step). Thereafter, butyl acetate was distilled off under reduced pressure at 90° C. to a solids concentration of 50%. The oil layer obtained was gradually cooled to 25° C., and a precipitated solid was filtered. Under reduced pressure, the solid obtained was dried by heating to 60° C. to obtain 190 g of a compound (1-1-1)-containing composition C1. From the results of ¹H-NMR and ¹³C-NMR analyses, the composition obtained was confirmed to contain compound (1-1-1).

The yield relative to 3, 4′-diaminodiphenyl ether was 68%.

An XRD measurement chart of the compound (1-1-1)-containing composition C1, which is the solid obtained, is shown in FIG. 2.

As a result of evaluation by XRD measurement, the composition C1 was shown to be a crystalline composition exhibiting crystallinity and having a degree of crystallinity of 95.5%.

For the compound (1-1-1)-containing composition C1 obtained, in a gel permeation chromatography analysis using a differential refractometer as a detector under the above conditions, the peak area of compound (1-1-1) relative to the peak area of all components detected (hereinafter referred to as the peak area percentage of compound (1-1-1)), that is, the purity of compound (1-1-1), was 92.9%. The composition was also shown to contain 7.1 area % of high-molecular-weight components derived from the synthesis step. A chart of this gel permeation chromatography analysis is shown in FIG. 3.

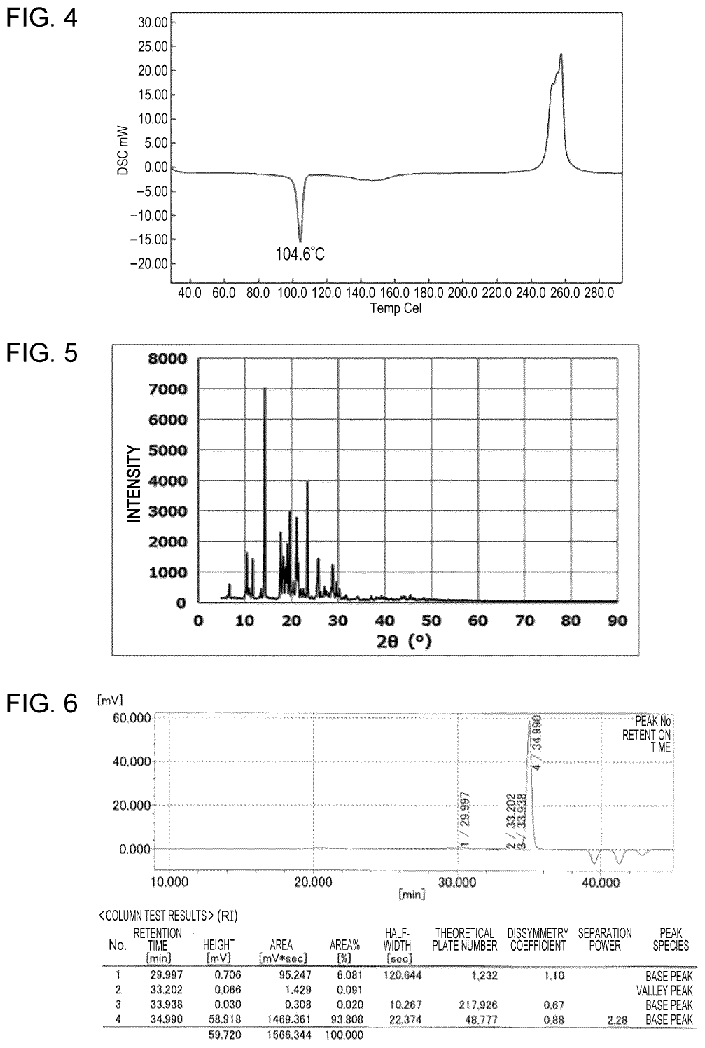

As a result of differential scanning calorimetry (DSC), the compound (1-1-1)-containing composition C1 was shown to be a crystal having a maximum endothermic peak temperature of 104.6° C. The DSC data is shown in FIG. 4.

The NMR data of the compound (1-1-1)-containing composition C1 obtained are shown.

¹H-NMR (400 MHZ) measurement (solvent: CDCl₃): 4.64 (s, 2H), 4.66 (s, 2H), 5.37 (s, 2H), 5.39 (s, 2H), 6.53 to 6.55 (ddd, 1H), 6.81 to 7.35 (m, 15H).

¹³C-NMR (400 MHZ) measurement (solvent: CDCl₃): 50.28, 50.36, 79.21, 80.14, 108.15, 110.60, 112.23, 114.69, 117.06, 120.20, 120.96, 126.85, 127.97, 128.33, 129.14, 130.20, 149.90, 151.27, 154.38, 159.06.

### Example 2

A solid of a compound (1-1-1)-containing composition C2 was obtained in the same manner as in Example 1 except that the amount of acetic acid added was 0.3 times the amount of 3,4′-diaminodiphenyl ether on a molar basis and the pH after washing with water was 6.

An XRD measurement chart of the compound (1-1-1)-containing composition C2, which is the solid obtained, is shown in FIG. 5.

As a result of evaluation by XRD measurement, the composition C2 obtained was shown to be a crystalline composition exhibiting crystallinity and having a degree of crystallinity of 95.8%.

The peak area percentage of compound (1-1-1), that is, the purity of compound (1-1-1), in the compound (1-1-1)-containing composition C2 obtained was 93.8%. The composition was also shown to contain 6.2 area % of high-molecular-weight components derived from the synthesis step. A chart of this gel permeation chromatography analysis is shown in FIG. 6.

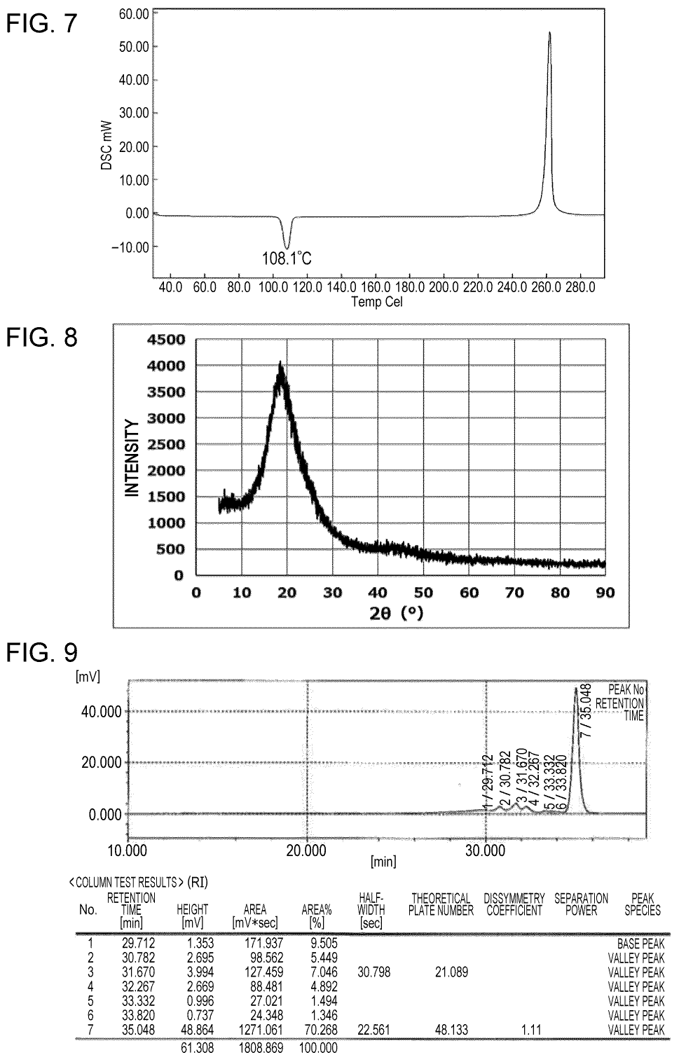

As a result of DSC, the compound (1-1-1)-containing composition C2 was shown to be a crystal having a maximum endothermic peak temperature of 108.1° C. The DSC data is shown in FIG. 7.

### Comparative Example 1: Production Method Described in PTL 2

In a four-necked flask equipped with a thermometer, a stirrer, and a condenser, 547 g (16.8 mol) of 92 wt % paraformaldehyde, 3336 g of toluene, and 394 g (4.19 mol) of phenol were loaded, and after the reaction vessel was purged with nitrogen, a solution prepared by dissolving 839 g (4.19 mol) of 3,4′-diaminodiphenyl ether, 839 g of toluene, and 394 g (4.19 mol) of phenol at 70° C. was intermittently added dropwise over 6 hours at 80° C. (at this time, the molar ratio of 3,4′-diaminodiphenyl ether, phenol, and paraformaldehyde was 1:2:4). Thereafter, stirring was performed at 82° C. for 18 hours. The reaction solution was analyzed by gel permeation chromatography to show that the peak area percentage of compound (1-1-1) was 70.2 area %.

After completion of the reaction, 1800 g of a 3% aqueous sodium hydroxide solution was added at 30° C. with stirring, and after stirring for 30 minutes, the resulting mixture was allowed to stand, and an aqueous layer was separated and removed. Thereafter, 2200 g of water was added to an oil layer with stirring at 30° C., and after stirring for 30 minutes, the resulting mixture was allowed to stand, and an aqueous layer was separated and removed. The procedure from the addition of water to the extraction of an aqueous layer was repeated four times.

From the oil layer obtained, toluene and phenol were removed by distillation under reduced pressure. The temperature and pressure during the distillation were gradually increased and decreased, finally reaching 90° C. and 1.5 kPa, respectively. A molten liquid of the composition containing compound (1-1-1) was extracted, solidified by cooling, and then pulverized to obtain 1383 g of a solid of a compound (1-1-1)-containing composition C3.

From the results of ¹H-NMR and ¹³C-NMR analyses, the compound (1-1-1)-containing composition C3 obtained was confirmed to contain compound (1-1-1).

An XRD measurement chart of the compound (1-1-1)-containing composition C3, which is the solid obtained, is shown in FIG. 8.

As a result of evaluation by XRD measurement, the composition C3 was shown to be a non-crystalline composition exhibiting amorphism and having a degree of crystallinity of 0%.

The peak area percentage of compound (1-1-1), that is, the purity of compound (1-1-1), in the compound (1-1-1)-containing composition C3 obtained was 70.3%. The composition was also shown to contain 29.7 area % of high-molecular-weight components derived from the synthesis step. A chart of this gel permeation chromatography analysis is shown in FIG. 9.

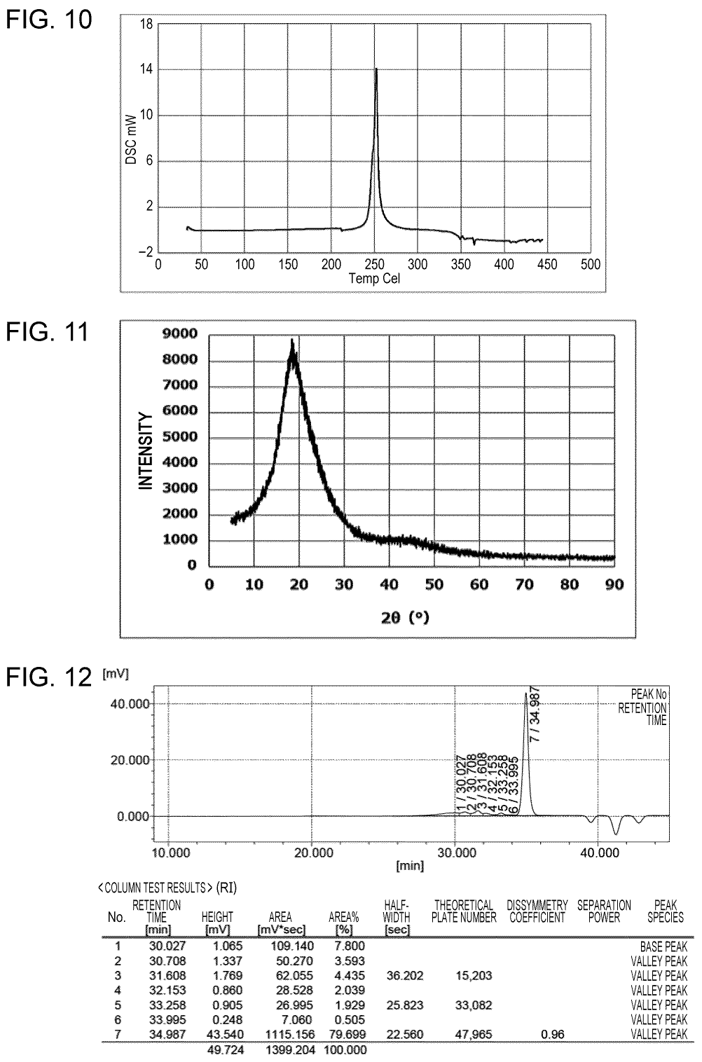

As a result of differential scanning calorimetry (DSC), the compound (1-1-1)-containing composition C3 was shown not to have a crystal structure because only an exothermic peak (curing reaction) was observed, and no endothermic peak (melting from a crystal phase) was observed, that is, no melting phase change was observed. The DSC data is shown in FIG. 10.

### Comparative Example 2

The compound (1-1-1)-containing composition C1 obtained in Example 1 and the compound (1-1-1)-containing composition C3 obtained in Comparative Example 1 were melt-mixed and cooled to obtain a solid of a compound (1-1-1)-containing composition C4.

An XRD measurement chart of the compound (1-1-1)-containing composition C4, which is the solid obtained, is shown in FIG. 11. As a result of evaluation by XRD measurement, the composition C4 was shown to be a non-crystalline composition exhibiting amorphism and having a degree of crystallinity of 0%.

The peak area percentage of compound (1-1-1), that is, the purity of compound (1-1-1), in the compound (1-1-1)-containing composition C4 obtained was 79.7%. The composition was also shown to contain 20.3 area % of high-molecular-weight components derived from the synthesis step. A chart of this gel permeation chromatography analysis is shown in FIG. 12.

### Comparative Example 3

The compound (1-1-1)-containing composition C1 obtained in Example 1 and the compound (1-1-1)-containing composition C3 obtained in Comparative Example 1 were melt-mixed at a weight ratio different from that in Comparative Example 2 and cooled to obtain a solid of a compound (1-1-1)-containing composition C5.

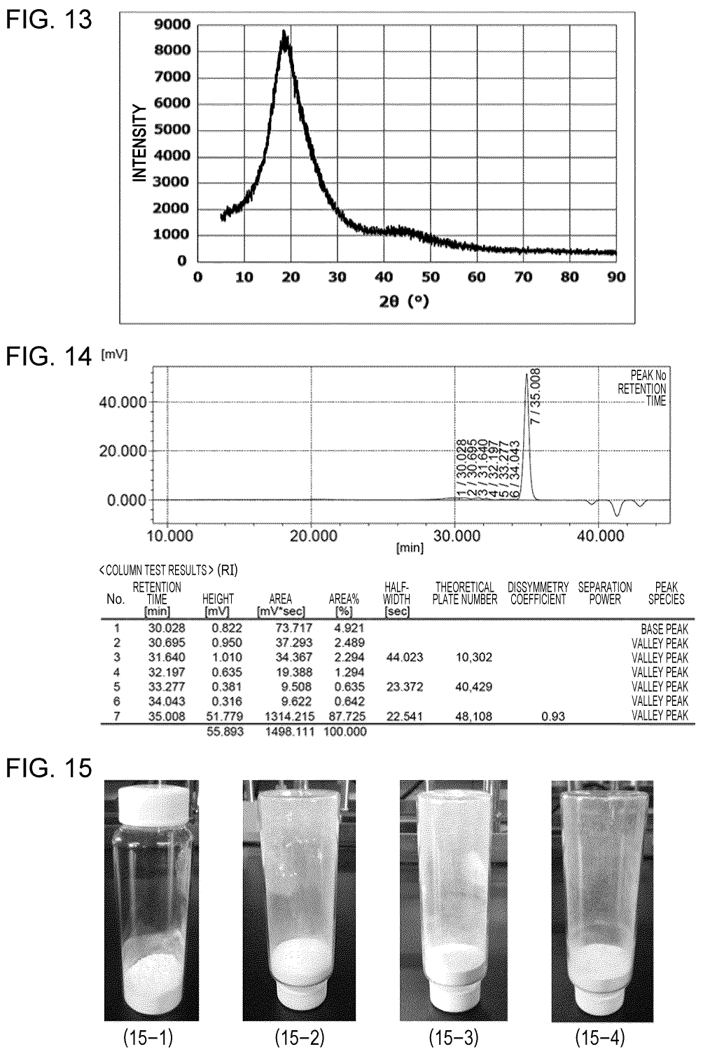

An XRD measurement chart of the compound (1-1-1)-containing composition C5, which is the solid obtained, is shown in FIG. 13. As a result of evaluation by XRD measurement, the composition C5 was shown to be a non-crystalline composition exhibiting amorphism and having a degree of crystallinity of 0%.

The peak area percentage of compound (1-1-1), that is, the purity of compound (1-1-1), in the compound (1-1-1)-containing composition C5 obtained was 87.7%. The composition was also shown to contain 12.3 area % of high-molecular-weight components derived from the synthesis step. A chart of this gel permeation chromatography analysis is shown in FIG. 14.

### Test for Assessing Storage Stability at High Temperatures and Handleability of Crystalline Composition

### (1) Composition C1 Obtained in Example 1 (Specific Example of Present Invention)

Three capped cylindrical glass sample bottles of 50 mL capacity and about 4 cm diameter each containing 10.00 g of the crystalline composition C1 obtained in Example 1 were prepared. A photograph of one of them in this state is shown in (15-1) in FIG. 15.

The sample bottles were respectively placed in water baths kept at 40° C., 50° C., and 60° C. and held for 24 hours. Thereafter, the sample bottles were inverted to check the fluidity of the crystalline composition C1. Photographs in this state are shown in (15-2) in FIG. 15 (kept at 40° C.), (15-3) in FIG. 15 (kept at 50° C.), and (15-4) in FIG. 15 (kept at 60° C.).

As a result, the crystalline composition C1 stored at each of the temperatures of 40° C., 50° C., and 60° C. hardly adhered to the bottoms of the sample bottles, and 9.95 g of the crystalline composition C1 fell on the cap portions of the inverted sample bottles.

The percentage of the crystalline composition C1 discharged from the sample bottles was 99.5%.

### (2) Composition C3 Obtained in Comparative Example 1

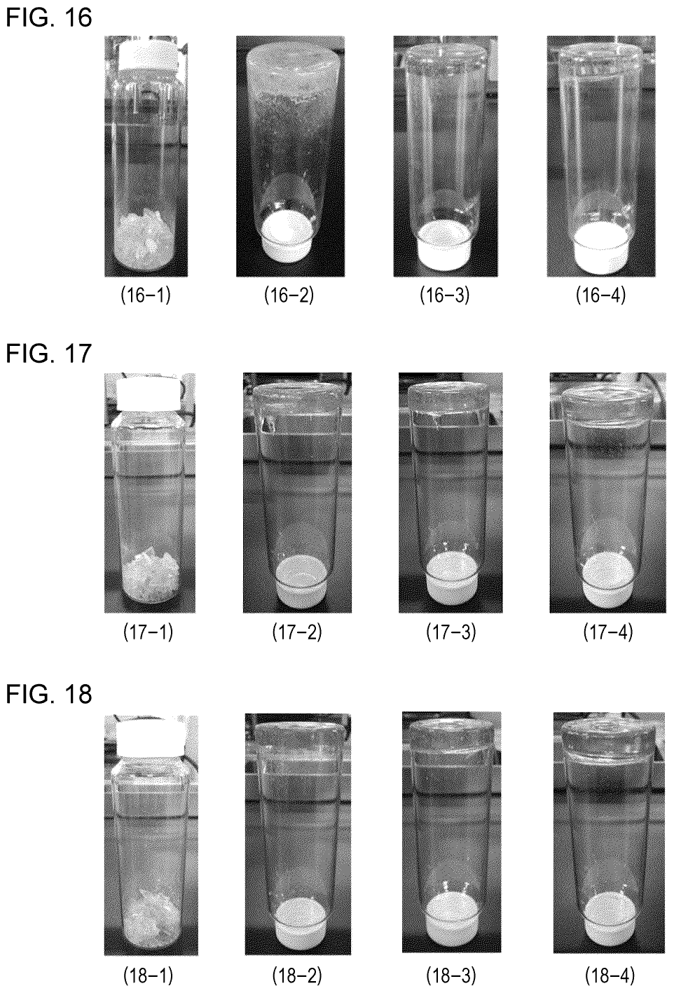

Next, using the composition C3 obtained in Comparative Example 1, the test was performed in the same manner as in (1) above to assess storage stability and handleability. A photograph of a state before the start of the test is shown in (16-1) in FIG. 16.

As a result, the samples stored at 40° C., 50° C., and 60° C. adhered in lumps to the bottoms of the sample bottles and did not fall even when inverted. Photographs in this state are shown in (16-2) in FIG. 16 (kept at 40° C.), (16-3) in FIG. 16 (kept at 50° C.), and (16-4) in FIG. 16 (kept at 60° C.).

The percentage of the composition C3 discharged from the sample bottles was 0.0%.

### (3) Composition C4 Obtained in Comparative Example 2

Next, using the composition C4 obtained in Comparative Example 2, the test was performed in the same manner as in (1) above to assess storage stability and handleability. A photograph of a state before the start of the test is shown in (17-1) in FIG. 17.

As a result, the samples stored at 40° C., 50° C., and 60° C. adhered in lumps to the bottoms of the sample bottles and did not fall even when inverted. Photographs in this state are shown in (17-2) in FIG. 17 (kept at 40° C.), (17-3) in FIG. 17 (kept at 50° C.), and (17-4) in FIG. 17 (kept at 60° C.).

The percentage of the composition of the composition C4 discharged from the sample bottles was 0.0%.

### (4) Composition C5 Obtained in Comparative Example 3

Next, using the composition C5 obtained in Comparative Example 3, the test was performed in the same manner as in (1) above to assess storage stability and handleability. A photograph of a state before the start of the test is shown in (18-1) in FIG. 18.

As a result, the samples stored at 40° C., 50° C., and 60° C. adhered in lumps to the bottoms of the sample bottles and did not fall even when inverted. Photographs in this state are shown in (18-2) in FIG. 18 (kept at 40° C.), (18-3) in FIG. 18 (kept at 50° C.), and (18-4) in FIG. 18 (kept at 60° C.).

The percentage of the composition of the composition C5 discharged from the sample bottles was 0.0%.

### (5) Results

The results of (1) to (4) show that the crystalline composition C1 obtained in Example 1, which is a specific example of the present invention, is greatly superior in storage stability at high temperatures and handleability to the composition C3 known in the art, which is amorphous, and even to the composition C4 and the composition C5, which are purer than the composition C3.

1. A crystalline composition comprising a benzoxazine compound represented by general formula (1): 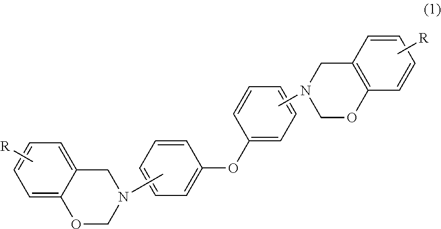
where each R is independently a hydrogen atom or a methyl group.

2. The crystalline composition according to claim 1, wherein a degree of crystallinity evaluated by X-ray diffractometry is 1% to 100%.

3. The crystalline composition according to claim 2, wherein the degree of crystallinity evaluated by X-ray diffractometry is 50% to 100%.

4. The crystalline composition according to claim 2, wherein the degree of crystallinity evaluated by X-ray diffractometry is 80% to 100%.

5. The crystalline composition according to claim 1, wherein in a gel permeation chromatography measurement using a differential refractometer as a detector, a peak area of the benzoxazine compound represented by general formula (1) is in a range of 80 area % to 100 area % relative to a peak area of all components detected.

6. The crystalline composition according to claim 1, wherein a maximum endothermic peak temperature determined by differential scanning calorimetry is 90° C. to 120° C.

7. A crystal of a benzoxazine compound represented by general formula (1-1), wherein a maximum endothermic peak temperature determined by differential scanning calorimetry is 90° C. to 120° C.: 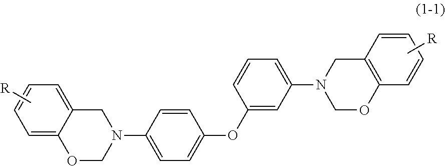
wherein each R is independently a hydrogen atom or a methyl group.

8. The crystal of the benzoxazine compound according to claim 7, wherein the benzoxazine compound represented by general formula (1-1) is a benzoxazine compound represented by formula (1-1-1):

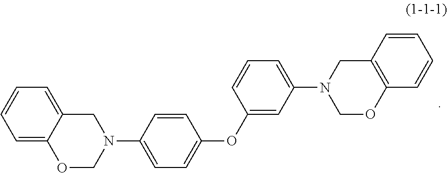

9. The crystal of the benzoxazine compound according to claim 7, wherein in a gel permeation chromatography measurement using a differential refractometer as a detector, a peak area of the benzoxazine compound represented by general formula (1-1) is in a range of 80 area % to 100 area % relative to a peak area of all components detected.

10. A method for producing a benzoxazine compound represented by general formula (1), the method comprising three reaction steps of a first step, a second step, and a third step:
the first step of reacting a diaminodiphenyl ether represented by general formula (2) and a hydroxybenzaldehyde represented by general formula (3) to obtain a compound represented by general formula (A): 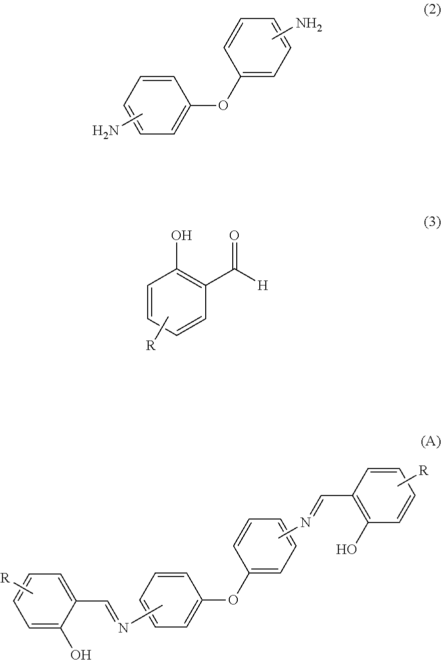
wherein each R is independently a hydrogen atom or a methyl group;
the second step of reducing the compound represented by general formula (A) to obtain a compound represented by general formula (B): 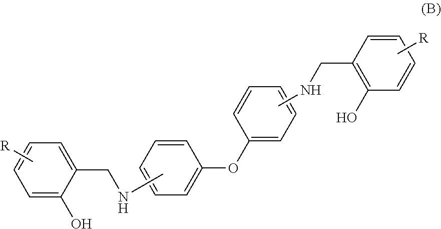
wherein each R is independently a hydrogen atom or a methyl group;
the third step of reacting the compound represented by general formula (B) and a formaldehyde to obtain a compound represented by general formula (1); 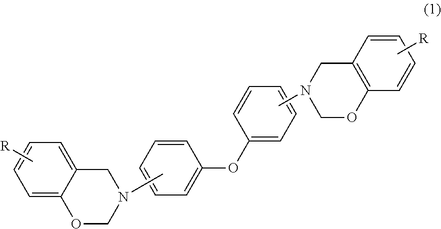
wherein each R is independently a hydrogen atom or a methyl group.

11. A compound represented by general formula (A-1): 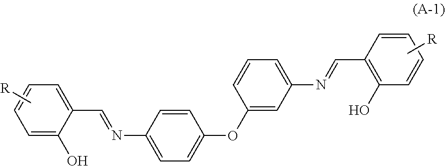
wherein each R is independently a hydrogen atom or a methyl group.

12. A compound represented by general formula (B-1): 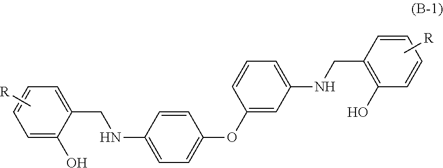
wherein each R is independently a hydrogen atom or a methyl group.
